(19) United States
(12) Patent Application Publication
(10) Pub. No.: US 2006/0025344 A1
(43) Pub. Date: Feb. 2, 2006

(54) Ghrelin receptor inverse agonists for regulation of feeding behaviors

(75) Inventors: Birgitte H. Lange, Copenhagen (DK); Thue W. Schwartz, Frederiksberg (DK); Thomas M. Frimurer, Copenhagen (DK); Oystein Rist, Vanlose (DK)

Correspondence Address: EDWARDS & ANGELL, LLP, P.O. BOX 55874, BOSTON, MA, 02205, US

(73) Assignee: 7TM Pharma A/S, Horsholm (DK)

(21) Appl. No.: 11/158,348

(22) Filed: Jun. 20, 2005

Related U.S. Application Data
(63) Continuation of application No. PCT/DK03/00924, filed on Dec. 20, 2003.

(30) Foreign Application Priority Data
Dec. 20, 2002 (DK) PA 2002 01983

(51) Int. Cl. A61K 38/22; C07K 14/575

(52) U.S. Cl. 514/13; 530/326

(57) ABSTRACT

Compounds of the invention act as inverse agonist ghrelin receptors. Some of the compounds of the invention may have both inverse agonistic and antagonistic properties as they both decrease or eliminate the constitutive activity of he ghrelin receptor and block the effect of ghrelin. Other preferred compounds of the invention have inverse agonistic properties but have little or no antagonistic activity. The compounds are suitable for medical and/or cosmetic use in connection with modulation of feeding behaviors, body composition and reduction of body mass. The invention also relates to methods for identifying inverse agonists for the ghrelin receptor and for monitoring the further development of such compounds.

### FIELD OF THE INVENTION

The invention relates to compounds that act as inverse agonists against ghrelin receptors. Some of the compounds of the invention may have both antagonistic and inverse agonistic properties as they both block the effect of ghrelin and decrease or eliminate the constitutive activity of the ghrelin receptor. Other preferred compounds of the invention have inverse agonistic properties but have little or no antagonistic activity. The compounds are suitable for medical and/or cosmetic use in connection with modulation of feeding behaviors, body composition and reduction of body mass. The invention also relates to methods for identifying inverse agonists for the ghrelin receptor and for monitoring the further development of such compounds.

### BACKGROUND OF THE INVENTION

Obesity is a disease with strongly increasing prevalence and it has reached epidemic proportions in the industrialized world. This disease is essentially characterized by an unbalance between energy intake and expenditure, which, without interference, leads to an ever increase in adipose tissue mass and body weight.

Obesity is associated not only with a social stigma, but also with decreased life span and numerous medical problems, including life-threatening chronic diseases such as coronary heart disease, hypertension, diabetes type II and certain types of cancer.

Dietary therapy often has a low success rate in the long run, and therefore there has been an increasing demand for pharmaceutical alternatives.

Appetite and energy intake are influenced by several hormonal effectors and neurotransmitters acting in the peripheral as well as the central nervous system. The hormones and neurotransmitters can be divided into those that act rapidly to influence individual meals, and those that act more slowly to promote the stability of body fat stores. Examples of long-term regulators are insulin and leptin, which both counteracts feeding and stimulates reduction in adipose mass. Examples of short-duration regulators are e.g. cholecystokinin, which is released from the gastrointestinal tract during eating and acts as a satiety signal, and ghrelin, which also is released from the GI tract but acts as an orexigenic hormone, which stimulates appetite and food intake. The present invention deals with the ghrelin system and how to interfere with this for treating obesity and related diseases.

The story of ghrelin, its receptor and synthetic compounds acting through this receptor unraveled in a unique “reverse” order. This is important for understanding why the high degree of ligand independent signalling and the use of inverse agonists for treatment of obesity and related disorders have first been discovered now.

In the eighties a synthetic hexa-peptide from a series of opioid-like peptides was found to be able to release growth hormone (GH) from isolated pituitary cells (Bowers et al., 1980). Since this action was independent of the growth hormone releasing hormone (GHRH) receptor, several pharmaceutical companies embarked upon drug discovery projects based on this hexa-peptide GH secretagogue (GHS) and its putative receptor. Several series of potent and efficient peptide as well as non-peptide GH secretagogues were consequently described in the mid nineties (Bowers et al., 1984; Patchett et al., 1995; Smith et al., 1993). However, first several years later was the receptor through which these artificial GH secretagogues acted eventually cloned and shown to be a member of the 7TM G-protein coupled receptor family (Howard et al., 1996; Kojima et al., 1999). But, first in 1999 was the endogenous ligand for this receptor, the hormone ghrelin finally discovered and surprisingly found to be produced in large amounts in endocrine cells in the stomach and only to a small extent centrally as originally expected (Bednarek et al., 2000).

Since the ghrelin receptor was so well known and believed to be so well-characterized when it was finally cloned, very little was in fact done to characterize it in general besides confirming that it had properties similar to those expected for the growth hormone secretagogue receptor as previously studied.

Moreover, after the cloning of the receptor calcium mobilization assays has been almost exclusively used to monitor signalling of this receptor since this signalling assay had become the industry standard for determining coupling through the Gq as well as several other signalling pathways. Unfortunately, it is very difficult or impossible to detect constitutive signalling when measuring intracellular calcium, which besides acute fluctuations during the initial phases of signalling is kept within strict limits within the cells through a number of mechanisms.

Ghrelin is a 28 amino acid peptide, which has a unique structure among peptide hormones as it is acylated at Ser3 usually with an n-octanyl moiety (Bednarek et al., 2000; Kojima et al., 1999). This post-translational modification is essential for the activity of the hormone—as mediated through the now classical 7TM G protein coupled ghrelin receptor—both in vitro and in vivo (Kojima et al., 1999; Nakazato et al., 2001; Tschop et al., 2000).

Plasma levels of ghrelin rise precipitously in the blood before meals, when the stomach is empty, and falls just as quickly after or during food consumption. Since i.v. or i.c.v administration of ghrelin increases food intake, it appears that the physiological role of ghrelin is to be a link or messenger between the stomach and the hypothalamus and the pituitary. A favored over-all mechanism is, that when the organism is getting ready for a meal, the CNS sends signals to the GI tract telling that a meal is about to be consumed in order to obtain information back about the status of the digestive process, state of distension etc. from the various chemical and mechanical sensors in the gut. Here, ghrelin could be an important hormonal messenger, which is sent back towards the CNS as a signal telling that there is no food in the stomach and that the GI tract is ready for a new meal. In such a paradigm it is clear that a blocker of the ghrelin receptor would be a very efficient anti-obesity agent, as it would block the meal initiating, appetite signal from the GI tract.

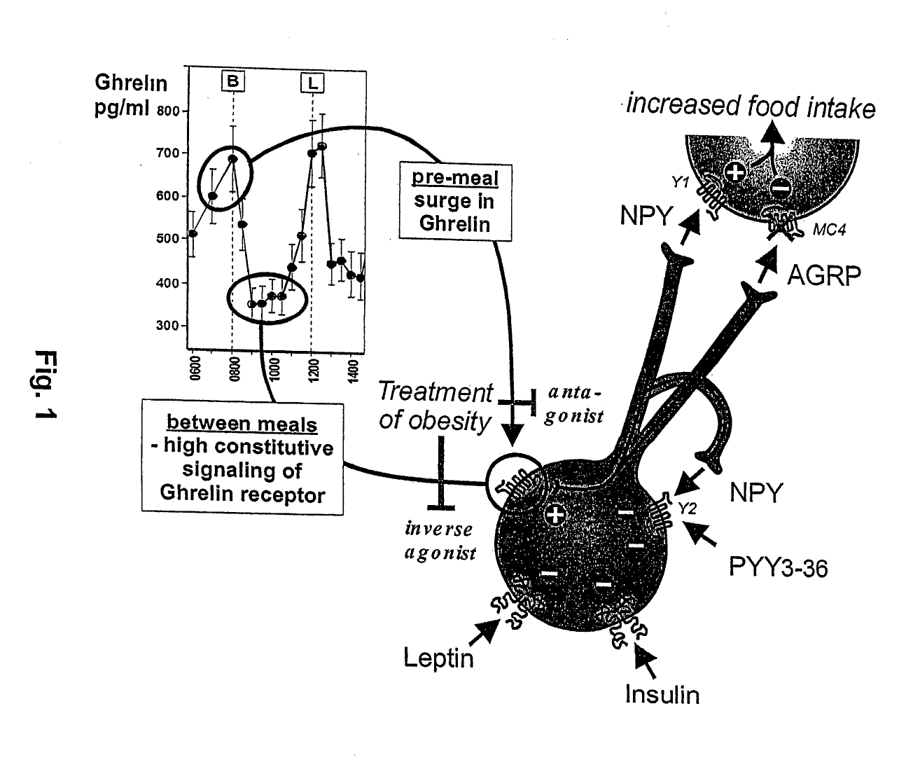

Centrally, ghrelin acts mainly on receptors expressed on NPY/AGRP producing cells in the arcuate nucleus of the hypothalamus (see FIG. 1). Functionally this has been demonstrated by use of antibodies and antagonists of NPY and AGRP which abolish the ghrelin induced feeding response (9). The NPY/AGRP neurons of the arcuate nucleus are very important parts of the stimulatory branch of the central control of food intake. Thus, ghrelin acts through stimulating the release of NPY and AGRP, which both work by stimulating neurons located mainly in the paraventricular nucleus (PVN). Here NPY acts by stimulating NPY receptors and AGRP acts as an antagonist and inverse agonists on melanocortin MC-3 and MC-4 receptors (the agonists for these are peptides derived from pro-opiomelanocortin (POMC)—mainly aMSH). Both of these downstream actions of ghrelin—i.e. stimulation of NPY receptors and inhibition of melanocortin receptors mainly in the PVN—result in increased food intake.

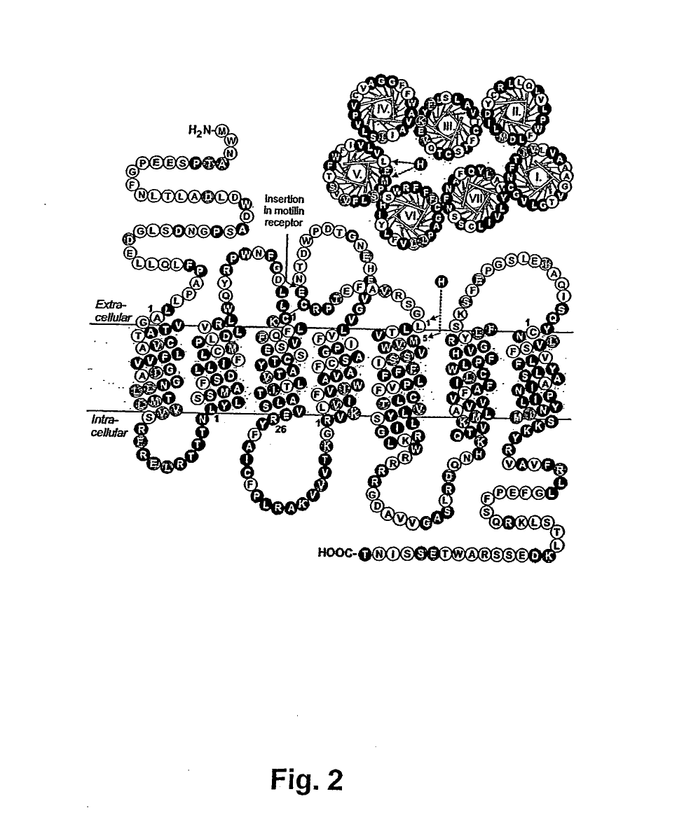

Interestingly, the ghrelin receptor was recently found to be expressed in large amounts also on afferent vagal neurons (Date et al., 2002; Asakawa et al., 2001). In accordance with this, the effect of peripheral administration of ghrelin on c-fos expression in NPY/AGRP neurons and the effect on feeding in rats is totally dependent on an intact vagal nerve, whereas the effect on GH secretion was only partially mediated through the proposed vagal afferent pathway (Date et al., 2002). These findings indicate that gastric vagal afferents may be a major pathway conveying ghrelins signalling from the stomach to the CNS. It could be noted that the closest homologue to the ghrelin receptor is the receptor for motilin (FIG. 2), which like ghrelin is a hormone secreted from the upper part of the gastrointestinal tract and which also interacts with the autonomic nervous system (Asakawa et al., 2001; Itoh, 1997). Ghrelin receptors are also found in the nucleus tractus solitarius in the brain stem in centers, which project to the hypothalamus. Thus, there appear to be at least three ways that the ghrelin signal to increase food intake etc. reaches the effector areas of the hypothalamus: 1) through action on ghrelin receptors on afferent vagal neurons which projects to the NTS and further on to the hypothalamus; and 2) through action on ghrelin receptors in the NTS; and 3) through direct action on ghrelin receptors in the arcuate nucleus especially on the NPY/AGRP neurons.

Importantly, in some animal experiments peripheral administration of ghrelin has even resulted in increase in body weight and fat mass as evaluated by DEXA scan under circumstances where the food intake was not even increased (Horvath et al., 2001; Tschop et al., 2000). This weight gain and increase in fat mass independent on an increased food intake may either be mediated by ghrelin receptors directly on the fat cells (Choi et al., 2003) or on the thyroid cells (Volante et al., 2003). In vitro studies have shown that ghrelin can act directly on the fat cells and inhibit the monoamine induced lipolysis and decrease apoptosis (Choi et al., 2003; Thompson et al., 2003). The ghrelin receptor is also highly expressed on thyroid cells but the functional consequences of ghrelin on these cells remains to be described. It is, however known that ghrelin administration decreases core body temperature in rodents, which indicates a decrease in the resting energy expenditure (Lawrence et al., 2002).

In total, ghrelin 1) stimulates food intake, 2) decreases energy expenditure, and 3) increases fat mass. Thus, the regulation of ghrelin function represents a very promising target in the field of obesity and it has been suggested that antagonists of the ghrelin receptor may be an important pharmacological option in the treatment of obesity.

The inventors of the present invention have found that the ghrelin receptor surprisingly is highly constitutively active and that this spontaneous signalling activity could be of physiological importance in its role in appetite control etc. The ligand-independent signalling of the ghrelin receptor is very high and similar to that displayed by one of the most vigorous constitutively active receptors yet known, the ORF-74 oncogene encoded by human herpes virus 8 (Bais et al., 1998; Rosenkilde et al., 1999).

Previously, different series of non-peptide, drug-like compounds have been developed for the ghrelin receptor. Importantly however, these are almost exclusively agonistic compounds, which were developed mainly aiming at increasing growth hormone (GH) secretion. Very few and only low potency antagonists have as yet been described for the ghrelin receptor probably due to the fact that people in the industry have been looking for agonists and not antagonists and have not at all been aware of the fact that the receptor is constitutively active and therefore have not tried to develop inverse agonists at all. The knowledge of the high constitutive activity opens for novel pharmaco-therapeutic opportunities in developing inverse agonist compounds for the ghrelin receptor for the treatment of a large variety of diseases or conditions.

### SUMMARY OF THE INVENTION

Accordingly, the present invention relates to inverse agonists of a ghrelin receptor for medical use.

In another aspect the invention relates to the use of inverse agonists for a ghrelin receptor for the preparation of a pharmaceutical composition for the treatment of overweight, obesity, type II diabetes and complications thereto. Since ghrelin as described above is a key stimulatory messenger in the control of appetite and the ghrelin receptor is highly constitutively active, an inverse agonist of the ghrelin receptor most certainly will have an inhibitory effect on food intake.

### DETAILED DESCRIPTION OF THE INVENTION

As described above the invention relates to inverse agonists of a ghrelin receptor for medical use.

The invention also relates to the use of inverse agonists of a ghrelin receptor for the preparation of a pharmaceutical composition for the treatment of overweight, obesity, type II diabetes and complications thereto.

Ghrelin is a key stimulatory messenger in the control of appetite and it has become clear from increasing knowledge about its role in the control system for appetite and energy homeostasis, that an antagonist for the ghrelin receptor would be beneficial in the treatment of obesity and related diseases. Such a compound would block the effect of the ghrelin hormone and would conceivably decrease the drive for initiation of a meal, which as described above appears to be the key role of the ghrelin hormone.

However, the discovery that the ghrelin receptor is signalling with high ligand-independent activity—i.e. that the receptor spontaneously is driving activity in for example the afferent vagal pathways, in the nucleus tractor solitarius in the brain stem, and in the NPY/AGRP neurons in the arcuate nucleus (FIG. 1) without any ghrelin hormone present, indicates that the ghrelin receptor—as such—is responsible for maintaining a signalling tone in the stimulatory branch of the control of food intake. This should be seen in the context that a large number of messenger systems such as leptin, insulin, aMSH, and PYY3-36 have the opposite effect as they act through inhibition of, for example the NPY/AGRP neurons (FIG. 1).

Thus, it appears that the constitutive signalling of the single most-important orexigenic hormonal pathway in the general control of appetite, i.e. the ghrelin receptor—through its ligand-independent activity—is keeping a high signalling tone in the stimulatory branch for the many inhibitory hormones and messengers to act on (FIG. 1). This ligand-independent ghrelin receptor activity appears to be the driver for our desire for, for example desserts and snacks at moments in time where the ghrelin hormone in fact is down at basal levels, i.e. after the surge in plasma levels of ghrelin, which allows for normal initiation of the main meals. Thus, an inverse agonist of the ghrelin receptor would take away the activating signalling “tone” in the stimulatory branch of the appetite control system and would therefore create a higher “appetite barrier” and eliminate the craving for, for example second-order of food, dessert and snacks and other types of non-needed food intake in between the main meals. This nippling behavior is known to be a major culprit in the development of obesity.

A pure inverse agonist, exemplified but not restricted to a compound such as [D-Arg¹, D-Phe⁵, D-Trp^{7,9}, Leu¹¹]-Substance P would according to the paradigm described above eliminate the drive for “second order of food, desserts and snacks”. However, since such a compound, which has little or no antagonistic properties—or rather perhaps a much lower potency as an antagonist than as an inverse agonists—would still allow ghrelin to deliver its GI tract derived appetite signal for normal food intake. This could be advantageous, as the organism requires a certain level of food intake even during a period where weight reduction should occur.

Nevertheless, part of the invention also relates to compounds which may act both as inverse agonists at the ghrelin receptor and thereby eliminate the desire to eat in between meals—and which may act also as antagonists at the receptor and thereby block the pre-meal appetite signal from the gut mediated through the ghrelin hormone. Such compounds having a double effect being both inverse agonists and antagonists would be expected to be stronger anti-appetite agents and could be used for persons with a greater need for weight reduction or to induce a weight reduction, whereas more pure inverse agonist for the ghrelin receptor may be particularly suited for maintaining a weight loss, which is a major problem in current treatments of obesity.

Considering that ghrelin acts as a modulator of the lipolysis in adipocytes and that the ghrelin receptor is highly constitutively active indicates that an inverse agonist or an antagonist of the ghrelin receptor will decrease the fat mass independently of its effect on appetite and food intake. Similarly, the effect of ghrelin on energy expenditure and the fact that the ghrelin receptor is highly constitutively active indicates that an inverse agonist or an antagonist of the ghrelin receptor will increase energy expenditure independent on its effect on appetite and food intake.

Even in the absents of changes in food intake ghrelin and ghrelin receptor agonists administration have been shown to modulate the body composition in favor of increased adipose tissue (Tschop et al., 2000; Horvath et al., 2001). It is not yet known whether this effect is mediated through hypothalamic neural circuits or whether it is mediated by the peripheral action of ghrelin on adipocytes or thyroid cells. However, it has been shown that the increase in adipose tissue mediated by ghrelin receptor agonists is independent of the NPY expression as shown in a NPY knock out mice model (Tschop et al., 2002). Based on these results it is expected that ghrelin receptor inverse agonists and antagonists will selectively decrease body fat mass independent on their effect on appetite and food intake.

Before going into details with the individual steps of the invention, in the following is given a list of specific terms used in the present text.

### DEFINITIONS

Throughout the text including the claims, the following terms shall be defined as indicated below.

A “ligand” as used herein is intended to mean a substance that either inhibits or stimulates the activity of a receptor and/or that competes for the receptor in a binding assay.

An “agonist” is defined as a ligand increasing the functional activity of a receptor.

An “inverse agonist” (also termed “negative antagonist”) is defined as a ligand decreasing the basal functional activity of a biological target molecule in this case the ghrelin receptor. Inverse agonism is a property of the ligand alone on the receptor. In the present context the term also includes partial inverse agonists, which only decreases the basal activity of the receptor to a certain level and not fully. It should be noted that certain compounds could be both an inverse agonist—in the absence of any hormone—and an antagonist—in the presence of the hormone.

An “antagonist” is defined as a ligand decreasing the functional activity of a biological target molecule by inhibiting the action of an agonist. In other words antagonism is a property of the ligand measured in the presence of a compound with higher signalling efficacy—i.e. usually a full agonist.

The “basal activity” or a “basal signalling activity” or “constitutive activity” or “constitutive signalling activity” of a receptor—in this case the ghrelin receptor—is defined as the signalling activity of the receptor in the absence of any ligand, i.e. hormone. This is also called the “ligand independent signalling”.

The term “IC50 for inverse agonism” intend to mean the concentration of a test compound (inverse agonist) required to obtain 50% maximum achievable inverse agonistic activity for that test compound—being an inverse agonist—i.e. the concentration required to decrease the activity of the constitutively activated ghrelin receptor by 50% of the maximum achievable decrease in activity (maximum achievable inverse agonistic response) provided by the inverse agonist. For a full inverse agonist IC50 for inverse agonism is the concentration of inverse agonist, which decreases the constitutive activity of the ghrelin receptor with 50%. For an 80% partial agonist it is the concentration of inverse agonist, which decreases the constitutive activity of the ghrelin receptor with 40%, i.e. down to 60% of the constitutive, basal activity.

The term “IC50 for antagonism” intend to mean the concentration of a test compound required to obtain 50% maximum achievable antagonistic activity for that test compound—being an inverse agonist which also is an antagonist—i.e. the concentration of test compound required to decrease the activity of the ghrelin receptor stimulated with a concentration of agonist, preferentially ghrelin, giving 90% of its maximal response down to 50% of the maximally achievable decrease obtainable with that test compound. The reason for using a 90% efficacious dose of agonist is that the “IC50 for antagonism” will be influenced by the dose of agonist, for example if higher doses of agonist is used this will mean that higher concentrations of antagonist is required to obtain the same degree of inhibition. For a test compound which can inhibit the signalling of the agonist stimulated ghrelin receptor fully, the “IC50 for antagonism” is the concentration required to inhibit the ghrelin stimulated activity down to 45% (i.e. 50% of the 90% obtained with the employed agonist concentration alone). For a test compound which can only inhibit the signalling of the agonist stimulated ghrelin receptor partially, the “IC50 for antagonism” is the concentration required to inhibit the ghrelin stimulated activity by 50% of the maximally achievable decrement in activity.

A “test compound” is intended to indicate a compound, which is capable of interacting with a receptor, in such a way as to binding to the receptor or to modify its biological activity.

In the present context the term “body mass index” or “BMI” is defined as body weight (kg)/height² (m²).

“Overweight” is intended to indicate a BMI in a range from about 25 to about 29.9.

“Obesity” is intended to indicate a BMI, which is at least about 30.

The term “efficient amount” as used herein means an amount of the peptide sufficient to attain the desired effect in the treatment of obesity in the animal, but not so large an amount as to cause serious side effects or adverse reactions.

One aspect of the invention provides inverse agonists identifiable by a method comprising the following steps:
a) contacting a ghrelin receptor with at least one test compound without the presence of an agonist for the ghrelin receptor, and
b) measuring any change in the basal activity of the ghrelin receptor and
c) identifying test compounds, that decreases the basal activity level of the ghrelin receptor with at least 1.0% such as e.g., at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%.

The invention also relates to a method for identifying inverse agonists of a ghrelin receptor, the method comprising step a), b) and c) as described above.

As follows from above the inverse agonists according to the invention are identifiable as compounds that are able to diminish the ligand-independent or constitutive signalling or spontaneous activity measured in cells expressing the ghrelin receptor. Thus, this is for example simply done by performing a dose-response experiment where the ghrelin receptor is exposed to increasing doses of the test compound and its signalling activity is measured, which—if the compound is an inverse agonist—will, gradually diminish in the presence of the compound.

One simple measure of the ability of a test compound to act as an inverse agonist is its potency measured as its IC50, i.e. the dose at which the compound is able to diminish the signalling of the receptor to half of the maximal effect of the compound. If a compound can totally eliminate the constitutive signalling (i.e. decrease the basal level activity with 100%), then it is called a full inverse agonist. Not all compounds are full inverse agonists as some compounds show lower efficacy as inverse agonists and only inhibit the signalling down to a certain level as described above. These are called partial inverse agonists.

Furthermore, in a specific embodiment an inverse agonist according to the invention has a ratio between IC50 for inverse agonism and IC50 for antagonism of the inverse agonist in a range of from about 1:1000 to about 1:10, such as, e.g., from about 1:750 to about 1:25, from about 1:500 to about 1:50, from about 1:400 to about 1:100, or from about 1:300 to about 1:200.

The ghrelin receptor used in an assay as described above can either be expressed endogenously on primary cells cultures, for example pituitary cells, or heterologously expressed on cells transfected with the ghrelin receptor. Whole cell assays or assays using membranes prepared form either of these cell types can be used depending on the type of assay.

As the ghrelin receptor is generally believed to be primarily coupled to the Gq signalling pathway, any suitable assay which monitor activity in the Gq/G11 signalling pathway can be used, for example: 1) an assay measuring the activation of Gq/G11 performed for example by measurement of GTPgS binding combined with, e.g., anti-Gaq or -11 antibody precipitation in order to increase the signal to noise ratio or 2) an assay which measure the activity of phopholipase C (PLC) one of the first down-stream effector molecules in the pathway, for example by measuring the accumulation of inositol phosphate which is one of the products of PLC (see examples for details of such an assay).

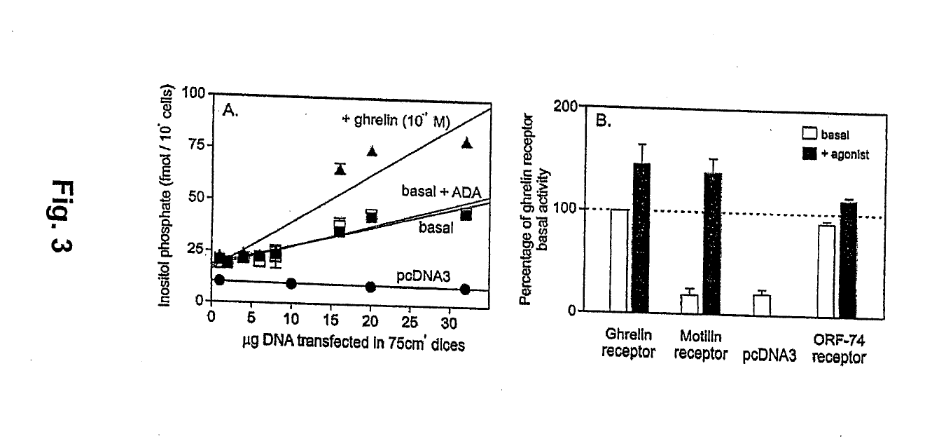

The traditional and dominating industrial standard assay for monitoring receptor signalling is based on the measurement of the mobilization of calcium from the intracellular stores. However, it is very hard to detect constitutive, ligand-independent signalling in a receptor using measurements of intracellular calcium as a read-out, due to the fact that intracellular calcium is kept within very stringent margins. The ligand-independent signalling of the ghrelin receptor has been overlooked until present conceivably due to the fact, that the receptor previously was studied almost exclusively in calcium mobilization assays. As described in the Examples (for example FIG. 3) the inventors have used for example inositol phosphate turnover as a measure of Gq signalling through the phospholipase C pathway, and it through such measurements was surprisingly found that ghrelin receptor in fact is highly constitutively active.

To be more specific, an inverse agonist according to the present invention has an inverse agonistic activity of about 20 μM or less, such as, e.g., about 15 μM or less, about 10 μM or less, about 7.5 μM or less, about 5 μM or less, about 2.5 or less, about 1 μM or less, about 750 nM or less, about 500 nM or less, about 400 nM or less, about 300 nM or less, about 200 nM or less, about 100 nM or less, about 75 nM or less, about 50 nM or less, about 25 nM or less, about 10 nM or less, about 5 nM or less, about 2.5 nM or less or about 1 nM or less, when measured in a ghrelin receptor-based signal-transduction assay, such as, e.g., a phosphatidylinositol turnover assay as described in the Examples.

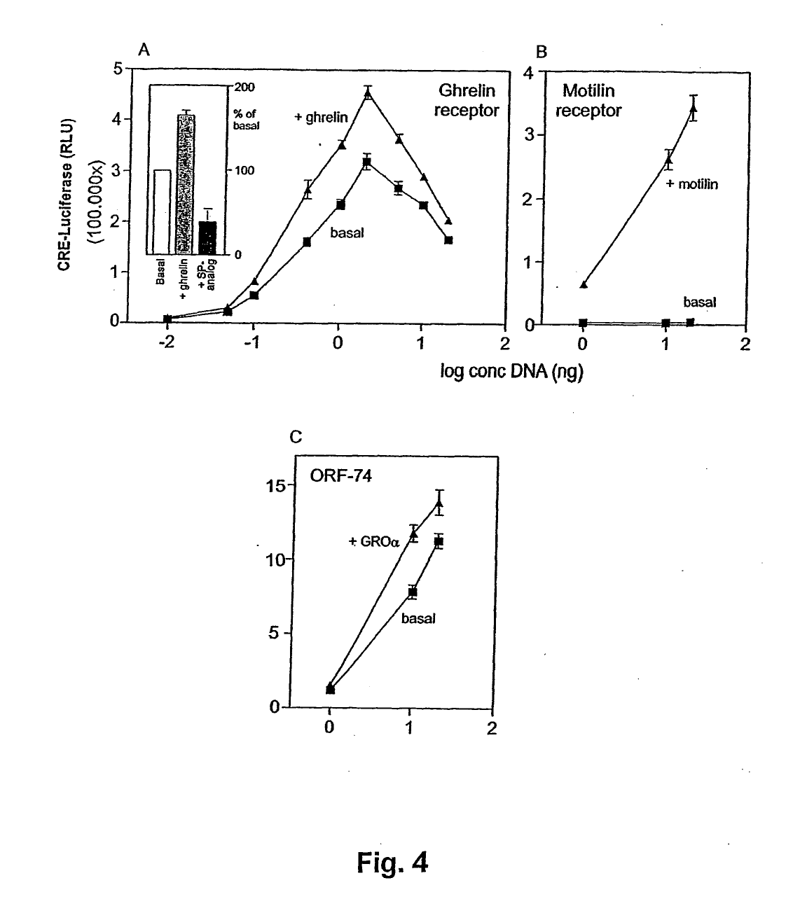

In the present invention it has been discovered that another assay, which is useful for detecting the ligand-independent signalling of the ghrelin receptor, is to measure cAMP responsive element (CRE) driven gene transcription. Such assays are commercially available, for example with luciferase as the reporter gene placed under the control of a series of CRE elements. As described in the Examples (FIG. 4) the ghrelin receptor drives CRE binding protein-dependent gene transcription with a high ligand-independent activity. The observed CRE activity appears to be of physiological importance as fasting induces an increase in the NPY level which appears to be mediated through an increase in CRE-dependent gene transcription as shown in transgenic mice expressing a CRE-lacZ construct (Shimizu-Albergine et al., 2001). Both the CRE-activation and the NPY up-regulation in response to fasting were clearly attenuated by leptin. However, in view of the strong effect of the ghrelin receptor on CRE-transcription discovered in the present invention (FIG. 4) and the fact that ghrelin is a major chemical messenger of fasting and appetite signals could suggest that the CRE-mediated up-regulation of NPY is regulated through the ghrelin receptor.

In the present invention it has also been discovered that other assays can be useful for detecting the ligand-independent signalling of the ghrelin receptor, i.e. assays measuring NFAT (Nuclear Factor of Activated T cell) -driven gene transcription. The results obtained with these assays further substantiate the discovery that the ghrelin receptor is characterized by a very high degree of spontaneous, constitutive signalling activity through multiple intracellular signalling pathways. Furthermore such assays can also be used to measure the effect and potency of inverse agonists and antagonists for the ghrelin receptor.

It will be obvious to a person knowledgeable in the art, that several different versions of the signalling assays described above as well as other signal transduction assays and other assays measuring for example mobilization of intracellular proteins such as arrestin can be used to measure the constitutive signalling activity of the ghrelin receptor and thereby to be used in a drug discovery process aiming at discovering and optimizing inverse agonists acting at the ghrelin receptor.

As mentioned above, an inverse agonist according to the invention may also have antagonistic activity. However, in a specific embodiment the inverse agonist is not an antagonist of a ghrelin receptor.

The ghrelin receptor for use in an antagonist assay may be expressed as described above for the inverse agonist assay. Whole cell assays or assays using cell membranes may be used. The signal transduction assays described above may also be used for measuring antagonism. In addition an assay, as mentioned above, which measure mobilization of calcium from the intracellular stores may be used. The assay may be performed by measuring fluctuations in intracellular calcium as such over time by one of many well-established methods.

A test compound can be probed for antagonistic activity on the ghrelin receptor by testing its ability to diminish or eliminate the signalling activity caused by stimulation of the ghrelin receptor by ghrelin or another ghrelin receptor agonist. In practice this is done by exposing the ghrelin receptor to the agonist in the absence and in the presence of the test compound and measuring signalling activity. Such experiments can be performed in various ways as for example a series of dose-response curves for the agonist performed in the presence of increasing doses of the test compound (a so-called Schild analysis) or simply as dose-response experiments of the test compound in the presence of a constant dose of the agonist, for example a sub-maximal stimulatory dose of the agonist, which stimulates signalling to for example 90% of the maximal response. A simple monitor of the ability of a test compound to act as an antagonist is to determine its potency measured as its IC50 for antagonism, i.e. the concentration at which it inhibits the agonist induced signalling by 50% of the maximally achievable decrement with that antagonist.

An inverse agonist according to the invention having antagonistic activity may have an antagonistic activity that is 10 μM or less such as, e.g., about 7.5 μM or less, about 5 μM or less, about 2.5 or less, about 1 μM or less, about 750 nM or less, about 500 nM or less, about 400 nM or less, about 300 nM or less, about 200 nM or less, about 100 nM or less, about 75 nM or less, about 50 nM or less, about 25 nM or less, about 10 nM or less, about 5 nM or less, about 2.5 nM or less or about 1 nM or less, when measured in a ghrelin receptor-based signal-transduction assay, such as, e.g., a phosphatidylinositol turnover assay as described in the Examples.

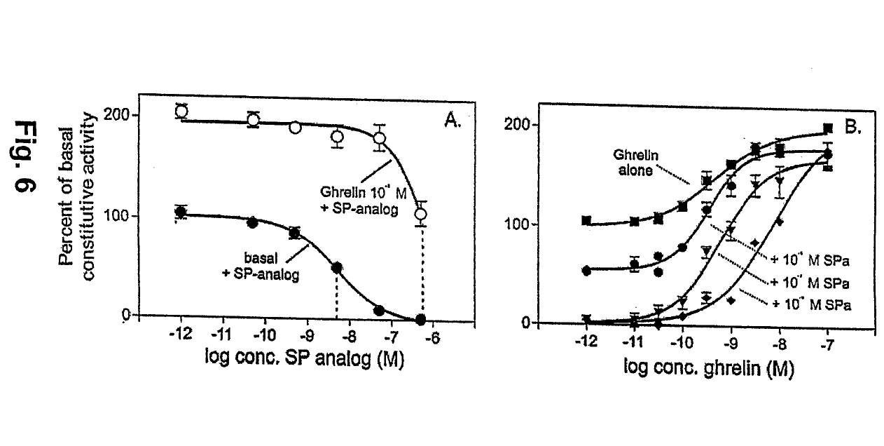

In order to compare the potency of a test compound as an inverse agonist and as an antagonist, respectively, we are in the current invention using mainly the IC50 for inverse agonism and IC50 for antagonism as defined above. This way of identifying the potency is unambiguous for an inverse agonist as it experimentally simply is the effect of the test compound on the receptor alone with no agonist present, which is probed. However, the IC50 value for antagonism is dependent on the dose of agonist used for stimulation of the receptor, i.e. the higher dose of agonist the higher IC50 for antagonism is obtained for a test compound. By using a 90% efficacious dose of the agonist the potency of the test compound as an antagonist may be underestimated. According to classical pharmacological principles, the potency of an antagonist is often determined through a so-called Schild analysis where a series of dose-response curves for the agonist are performed in the presence of increasing doses of the antagonist (see Examples, FIG. 6). The potency is in this way expressed for example as a pA2 value, which is the negative logarithm to base 10 of the concentration of the antagonist—provided it is a competitive antagonist—that shifts the concentration-response curve of an agonist two-fold to the right. This pA2 value corresponds closely to the pKB, which is the negative logarithm to the base 10 of the equilibrium dissociation constant of the—competitive—antagonist. Certain preferred compounds of the present invention are such which have a higher potency as inverse agonists than as antagonists (see below), these are for convenience defined as compounds for which the IC50 for inverse agonism is for example around 10 or more fold lower that the IC50 for antagonism.

Accordingly, in a specific embodiment an inverse agonist according to the invention having both inverse agonistic and antagonistic activity has a ratio between IC50 for inverse agonism and IC50 for antagonism of the inverse agonist in a range of from about 1:10 to about 1:0.01, such as, e.g., from about 1.8 to about 1:0.025, from about 1:6 to about 1:0.05, from about 1:4 to about 1:0.075, from about 1:2 to about 1:0.1, from about 1:1 to about 1:0.25, or from about 1:0.75 to about 1:0.5.

In one embodiment of the invention the test compound is a pure inverse agonist on the ghrelin receptor or rather a compound with a higher potency as an inverse agonist than as an antagonist. Such compounds should have IC50 values for inverse agonism, which are 10-fold or more lower than their IC50 values for antagonism. This can be exemplified by the [D-Arg¹, D-Phe⁵, D-Trp^{7,9}, Leu¹¹]-Substance P compound which as shown in FIG. 6A is approx. 100 fold more potent as an inverse agonist in inhibiting the constitutive signalling by the ghrelin receptor than as an antagonist in inhibiting the ghrelin stimulated signalling. As shown in FIG. 6B, Schild-type analysis (not classical as we here are dealing with the more complex effect of an inverse agonist which also is a low potency antagonist) demonstrates that the [D-Arg¹, D-Phe⁵, D-Trp^{7,9}, Leu¹¹]-Substance P compound decreases the spontaneous, constitutive signalling of the ghrelin receptor at low doses, which do not shift the dose-response curve for ghrelin to the right.

For practical reasons a compound can have such a low potency as an antagonist that it cannot be determined with the assay used and such a compound will then be designated as an inverse agonist which is not an antagonist. Such compounds also belong to the class of compounds defined as pure inverse agonists according to the invention.

In another embodiment of the invention the compound is both an inverse agonist and an antagonist, which means that the difference in its IC50 for inverse agonism and for antagonism is less than 10-fold. The IC50 for inverse agonism and for antagonism can even be the same or the IC50 for antagonism can be within 10-fold lower than the IC50 for inverse agonism. Such compounds, which all will be considered to be both inverse agonists and antagonists, are part of the invention and could be particular useful for treatment of obesity where the intenbon is both to inhibit the appetite between meals—especially performed by the inverse agonistic property of the compound—and during meals—especially performed by the antagonistc property of the compounds as presented and discussed above.

The inverse agonists according to the invention may be peptides. As shown in the invention (see Examples), [D-Arg¹, D-Phe⁵, D-Trp^{7,9}, Leu¹¹]-Substance P is a potent and highly efficacious inverse agonist for the ghrelin receptor as the compound at nano-molar concentrations inhibits the signalling down to that observed in cells not expressing the ghrelin receptor. However, this particular peptide is probably not very optimal as a general pharmacological tool or drug candidate since it at micromolar concentrations also has effects on the tachykinin NK1, i.e. the substance P receptor and at such high concentrations even affects a number of other receptors including the gastrin releasing peptide (GRP or bombesin) receptor. However, the substance P analog indicates that peptides can be discovered and developed to act as inverse agonists on the ghrelin receptor.

Di-peptide libraries based on this and similar substance P analogs have proven to be useful starting points for the development of non-peptide antagonists for several types of peptide receptors. [D-Arg¹, D-Phe⁵, D-Trp^{7,9}, Leu¹¹]-Substance P has a very interesting molecular pharmacological phenotype as it is a rather pure, high affinity inverse agonist with a low potency as an antagonist (FIG. 6).

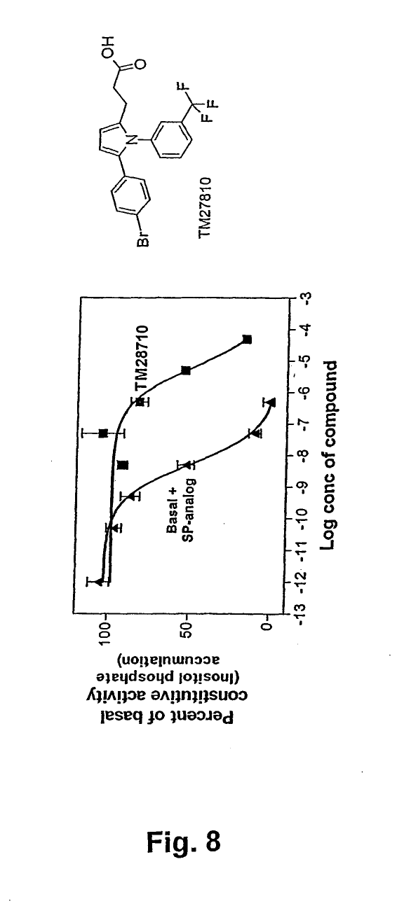

One preferred embodiment of the invention relates to inverse agonists, which are non-peptide compounds, i.e. small organic compounds with little or no chemical resemblance to peptides. Such compounds are often better drugs than peptides as they for example often can be administered orally successfully. The discovery of the non-peptide compound TM27810, which efficiently decreases the constitutive signalling activity of the ghrelin receptor, illustrates that not only peptides such as the substance P analog, but also non-peptide compounds can act as inverse agonists on the ghrelin receptor. TM27810 was discovered as a hit or lead compound in a small, selected, i.e. target-customized chemical library and is of relatively low potency as compared to the substance P analog (FIG. 8). However, it will be well known to the person knowledgeable in the art that chemical modifications of such a compound or other similar lead compounds can increase their affinity and potency and that compounds with appropriate high potency and appropriate pharmacokinetic properties can be developed on the basis of such lead compounds through well established medicinal chemical approaches.

Previously, different series of non-peptide, drug-like compounds have been developed for the ghrelin receptor. However, these were almost exclusively agonistic compounds, which were developed mainly aiming at developing drugs for increasing growth hormone (GH) secretion. Very few antagonists and only of low, i.e. micromolar affinity have as yet been described for the ghrelin receptor probably due to the fact that people in the industry have been looking for agonists and not antagonists. Importantly due to the fact that the constitutive activity of the ghrelin receptor was not previously recognized no attempts has been made to develop inverse agonists for the ghrelin receptor. The fact that non-peptide agonists for the ghrelin receptor previously with success have been discovered and developed into drug candidates indicates that structurally similar—or structurally distinct but still non-peptide compounds—can be developed which are inverse agonists or are both antagonists and inverse agonists. It will be well known to the person knowledgeable in the field that chemical modifications of an agonist can turn it into being an antagonist or an inverse agonist and the other way around.

Furthermore, the inverse agonists according to the invention may be antibodies, for example human or humanized antibodies. The ghrelin receptor belongs to the 7TM G protein coupled receptor family and it is well known that antibodies are not all that easy to develop against this class of membrane proteins. Antibodies may be developed against the ghrelin receptor and such antibodies, which will bind to the receptors, can act as antagonists, agonists or as inverse agonists. An antibody which act as an inverse agonist and which may or may not also be an antagonist could in some cases be preferred as a compound to treat obesity as opposed to a small molecule compound due to the long duration of the action of a antibodies in general.

Compounds that are inverse agonist may be identified by use of the following method according to the invention. This method comprises
a) contacting a ghrelin receptor with at least one test compound without the presence of an agonist for the ghrelin receptor, and
b) measuring any change in the basal activity of the ghrelin receptor
c) identifying test compounds, that decreases the basal activity level of the ghrelin receptor with at least 10%, such as e.g., at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%.

As mentioned hereinbefore, the compounds have utility in medicine. Accordingly, one aspect of the invention relates to a method for modulating by inverse agonism the activity of a ghrelin receptor by contacting the receptor comprising administering to a subject such as a mammal including a human with an effective amount of an inverse agonist according to the invention.

As described above the ghrelin receptor is considered to be a key regulator of food intake and energy expenditure and even of fat mass independent of its effects on food intake. Thus, by inhibiting the activity of the ghrelin receptor by inverse agonists acting for example on afferent vagal neurons, and/or on neurons in the NTS in the brain stem, and/or on the NPY/AGRP-expressing neurons in the hypothalamus, and/or on adipocytes, and/or on thyroid cells it is expected that the appetite will be inhibited, food intake will be decreased, energy expenditure decreased through an increased energy consumption especially increased lipolysis in the fat tissue.

Importantly, recently (i.e. after the submission of the priority application) the [D-Arg¹, D-Phe⁵, D-Trp^{7,9}, Leu¹¹]-Substance P peptide has been tested in vivo in mice, under the assumption that it was a ghrelin receptor antagonist (Asakawa et al., 2003). In the present invention it has been demonstrated that the potency of this peptide as an inverse agonist is approx. 100 fold higher than its potency as an antagonist. Repeated administrations of the peptide were performed for six days in normal and in ob/ob obese mice. [D-Arg¹, D-Phe⁵, D-Trp^{7,9}, Leu¹¹]-Substance P decreased energy intake in both the lean mice, in mice with diet induced obesity, as well as in ob/ob obese mice. The peptide also reduced the rate of gastric emptying, which is an important additional observation since this in itself will decrease food intake/meal size. Importantly, the repeated administrations of the peptide decreased body weight gain and also improved glycaemic control in the obese ob/ob mice. Since the [D-Arg¹, D-Phe⁵, D-Trp^{7,9}, Leu¹¹]-Substance P is an efficacious, i.e. full inverse agonist which has a much higher potency as inverse agonist than as an antagonist, it is argued that a major part if not all of the effect of the peptide in vivo on food intake and body weight gain is caused by the inverse agonist properties of the peptide, which obviously was unknown to the authors of the paper when it was written.

The invention relates to a method for modifying feeding disorders and/or treating and/or prevention diseases caused by feeding disorders, the method comprising administering to a mammal in need thereof an efficient amount of an inverse agonist of a ghrelin receptor according to the invention. An amount of an antagonist of a ghrelin receptor may also be applied.

The inverse agonist and/or antagonist of a ghrelin receptor may also be used to suppress hunger or reduce energy intake of a mammal or reduce body mass, to treat or prevent overeating including bulimia, bulimia nervosa, overweight and/or obesity, to treat or prevent Syndrome X (metabolic syndrome) or any combination of obesity; to treat or prevent insulin resistance, dyslipidemia, impaired glucose tolerance or hypertension; or to treat or prevent Type II diabetes or Non Insulin Dependent Diabetes Mellitus (NIDDM). Whenever relevant, the use may be medical as well as cosmetic. The latter is of specific importance concerning reduction of body mass, suppression of hunger and energy intake etc.

Use of the inverse agonists according to the invention may be supplemented by administration (before, concomitantly or after) simultaneously or sequentially of a further therapeutically or prophylactically active substance such as, e.g., an antagonist of a ghrelin receptor.

The invention also provides cosmetic and pharmaceutical compositions comprising an inverse agonist of a ghrelin receptor. Whenever relevant, the particulars and details described above under the use or compound aspect of the present invention may apply mutatis mutandis to the other aspects of the invention. In addition the invention relates to a method for the preparation of a pharmaceutical composition comprising an inverse agonist of a ghrelin receptor identifiable by a method as described above, the method for preparation comprising admixing the inverse agonist with one or more pharmaceutically acceptable excipients.

Furthermore, the invention provides a pharmaceutical composition comprising an inverse agonist of the ghrelin receptor or a pharmaceutical acceptable salt of the inverse agonist together with a pharmaceutical acceptable excipient. The inverse agonist of the ghrelin receptor may present in the pharmaceutical preparation in an amount sufficient to decrease the basic activity level of the ghrelin receptor with at least 10%, such as, e.g., at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100% as evidenced by testing the pharmaceutical composition in in vitro signalling assay described above, for example an assay using a cell line expressing the human ghrelin receptor and measuring for example IP turnover or CRE-driven gene transcription. Nornally, the inverse agonist of the ghrelin receptor constitutes from about 1 to about 95% w/w of a composition of the invention.

The pharmaceutical or cosmetic composition according to the invention may be for enteral and/or parenteral use, and may be administered to the mammal by any convenient administration route such as, e.g., the oral, buccal, nasal, ocular, pulmonary, topical, transdermal, vaginal, rectal, ocular, parenteral (including inter alia subcutaneous, intramuscular, and intravenous), route in a dose that is effective for the individual purposes. A person skilled in the art will know how to choose a suitable administration route.

The pharmaceutical or cosmetic composition comprising a compound according to the invention may be in the form of a solid, semi-solid or fluid composition.

The solid composition may be in the form of tablets such as, e.g. conventional tablets, effervescent tablets, coated tablets, melt tablets or sublingual tablets, pellets, powders, granules, granulates, particulate material, solid dispersions or solid solutions.

A semi-solid form of the composition may be a chewing gum, an ointment, a cream, a liniment, a paste, a gel or a hydrogel.

The fluid form of the composition may be a solution, an emulsion including nano-emulsions, a suspension, a dispersion, a liposomal composition, a spray, a mixture, a syrup or a aerosol.

Fluid compositions, which are sterile solutions or dispersions can utilized by for example intraveneous, intramuscular, intrathecal, epidural, intraperitoneal or subcutaneous injection of infusion. The compounds may also be prepared as a sterile solid composition, which may be dissolved or dispersed before or at the time of administration using e.g. sterile water, saline or other appropriate sterile injectable medium.

Other suitable dosages forms of the pharmaceutical compositions according to the invention may be vagitories, suppositories, plasters, patches, tablets, capsules, sachets, troches, devices etc.

The dosage form may be designed to release the compound freely or in a controlled manner e.g. with respect to tablets by suitable coatings.

The pharmaceutical composition may comprise a therapeutically effective amount of a compound according to the invention.

The pharmaceutical or cosmetic compositions may be prepared by any of the method well known to a person skilled in pharmaceutical or cosmetic formulation.

In pharmaceutical or cosmetic compositions, the compounds are normally combined with a pharmaceutical excipient, i.e. a therapeutically inert substance or carrier.

The carrier may take a wide variety of forms depending on the desired dosage form and administration route.

The pharmaceutically or cosmetically acceptable excipients may be e.g. fillers, binders, disintegrants, diluents, glidants, solvents, emulsifying agents, suspending agents, stabilizers, enhancers, flavors, colors, pH adjusting agents, retarding agents, wetting agents, surface active agents, preservatives, antioxidants etc. Details can be found in pharmaceutical handbooks such as, e.g., Remington's Pharmaceutical Science or Pharmaceutical Excipient Handbook.

The invention also relates to the use of an inverse agonist according to the invention or a pharmaceutically acceptable salt thereof for the manufacture of a cosmetic composition for reducing body weight.

Furthermore, the invention relates to the use of an inverse agonist according to the invention or a pharmaceutically acceptable salt thereof for the manufacture of a pharmaceutical composition for i) modifying the feeding behavior of a mammal, ii) suppressing hunger or reducing energy intake of a mammal, or for any other of the above-mentioned conditions.

A pharmaceutically composition of the invention contains a suitable dose of the inverse agonist. The composition may also contain an antagonist to a ghrelin receptor or any other suitable therapeutically and/or prophylactically active substances. A person skilled in the art will know how to determine an efficient daily dose and, optionally, split this dose in 2-6 administrations daily. However, normally the daily dose is in a range of 0.1 mg to 500 mg daily.

### LEGENDS

FIG. 1 shows a schematic overview of the function of the ghrelin receptor in the NPY/AGRP neurons in the stimulatory branch of the hypothalamic centre for control of appetite and food intake.

An NPY/AGRP expressing neuron located in the arcuate nucleus is shown with the main hormonal and transmitter inputs. At the top is indicated a target neuron, which could be for example a corticotrophin releasing hormone (CRF) or gastrin releasing peptide (GRP or mammalian bombesin) neuron, located in the paraventricular nucleus. In this “effector” centre of the hypothalamus information from several other centres are integrated and information is conveyed to the rest of the CNS. It should be noted that ghrelin—coming either as a hormone from the gastrointestinal tract or as a neuronal transmitter—acting through the ghrelin receptor is the main, dominating stimulatory input to this system. Several other messenger systems act through inhibiting this system, for example: leptin from adipose tissue, insulin from the pancreas, and PYY3-36 from the distal GI tract acting on presynaptic Y2 receptors, which also is the target for NPY. Thus the direct line of stimulation in this system is ghrelin acting on the ghrelin receptor stimulating the release of NPY acting on NPY Y1/Y5 receptors and AGRP acting as an antagonist/inverse agonist on melanocortin MC-4 receptors both of which are leading to increased food intake. In the present invention it is discovered that the ghrelin receptor is signalling with a high degree of ligand-independent activity, which will give a high basal stimulatory tonus in the stimulatory branch of the control of food intake, i.e. a high stimulatory signalling tone upon which the various inhibitory systems could work. In view of the fact that the appetite stimulating hormone ghrelin is secreted mainly just prior to a meal (indicated by the inset showing meal related fluctuations in plasma ghrelin levels; B=breakfast, L=lunch) it is clear that ghrelin receptor antagonists should be beneficial in the treatment of obesity by blocking meal-associated food-intake. Since plasma levels of ghrelin return towards basal levels during and at the end of a meal it should be obvious that a ghrelin receptor inverse agonist, which will take away the basal stimulatory tone in this stimulatory branch of food intake will be beneficial for the treatment of obesity by taking away especially the basal stimulatory drive for “second order meals”, desserts, and snacks—i.e. nibbling behavior.

FIG. 2 is a serpentine and helical wheel diagram of the ghrelin receptor.

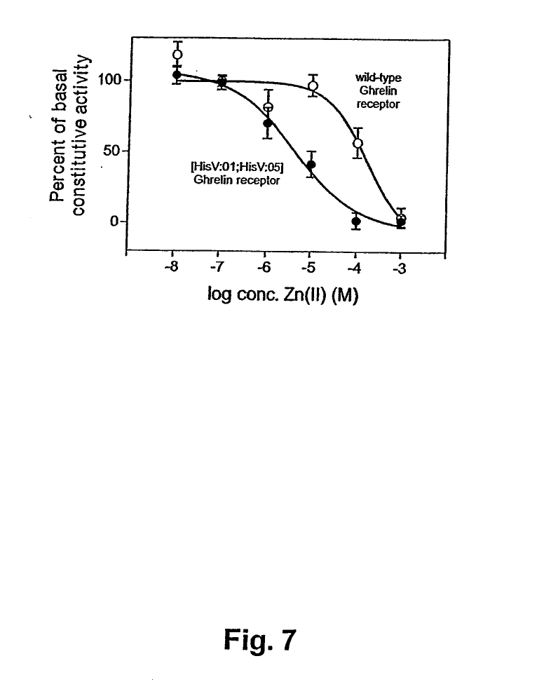

Residues, which are identical (white on black) or structurally conserved (white on grey) between the ghrelin and its closest homologue, the motilin receptor, are highlighted. The position in the extracellular loop 2 of an unusually long insertion of 39 amino acids, which is not found in the ghrelin receptor, is shown by an arrow. The histidine residues introduced as a bis-His metal ion site in the extracellular part of the fifth transmembrane segment are indicated with a dotted arrow. See example 4, FIG. 7 for effect of the non-peptide compound, Zn(II) as an inverse agonist on the ghrelin receptor through binding to this metal-ion site.

FIG. 3 illustrates the constitutive signalling of the ghrelin receptor as determined by analysis of inositol phosphate turnover.

Left panel: Gene-dosing experiments with the ghrelin receptor in transiently transfected COS-7 cells: basal constitutive activity (filled squares), constitutive activity after incubation in 30 min with adenosine deaminase (ADA) to eliminate a potential effect of adenosine in the system (open squares) compared to the ghrelin agonist stimulated, increased activity (filled triangles) and the lack of activity in cells transfected with the empty vector pcDNA3 (full circles). Data are mean±S.E. of three independent experiments made in triplicate. Right panel: Comparison of the basal constitutive activity and the agonist stimulated activity of the ghrelin receptor, the control motilin receptor and the well characterized, known constitutively active ORF-74 receptor from human herpes virus 8. Data are mean±S.E. of three independent experiments made in triplicate.

FIG. 4 shows the constitutive induction of cAMP responsive element (CRE) gene transcriptional activity by the ghrelin receptor (panel A) and by the ORF-74 receptor (panel C) but not by the control motilin receptor (panel B).

The ligand-indpendent, basal signalling activities of the three receptors (square symbols) and the signalling in the presence of a maximal dose of the relevant full agonist: ghrelin, motilin and GROα respectively (triangle symbols) was measured by a CREB-luciferase reporter assay in gene dosing experiments resulting in increasing receptor expression in transiently transfected HEK 293 cells (for details see Example 2—Experimental procedures). In the insert in panel A is shown the effect of ghrelin (10⁻⁶ M) and of [D-Arg¹, D-Phe⁵, D-Trp^{7,9}, Leu¹¹]-Substance P (10⁻⁶ M) on the basal CREB-luciferase activity in cells transfected with 2 ng ghrelin receptor DNA. Shown are representative experiments out of at least four independent experiments performed in quadruplicates. RLU—relative light units, as measured in a Packard TopCounter (5 secs/well).

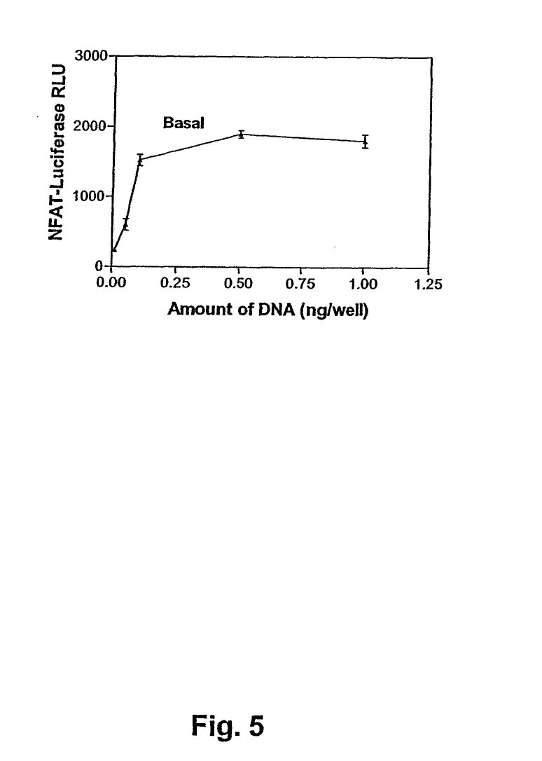

FIG. 5 shows the ligand independent induction of nuclear factor of activated T cell (NFAT) gene transcription activity by the ghrelin receptor.

Increasing constitutive, basal signalling of the receptor through the NFAT pathway was measure in gene-dosing experiments giving increasing receptor expression in transiently transfected HEK 293 cells (for details see Example 2—Experimental procedures).

FIG. 6 shows the effect of [D-Arg¹, D-Phe⁵, D-Trp^{7,9}, Leu¹¹]-Substance P as an inverse agonist on the constitutive activity (full circle) and as an antagonist on the ghrelin stimulated inositol phosphate turnover (open circle).

Panel A: The IC₅₀ for antagonism for [D-Arg¹, D-Phe⁵, D-Trp^{7,9}, Leu¹¹]-Substance P acting as an antagonist against ghrelin (10⁻⁸ M) stimulated signalling was 630±20 nM, whereas its IC₅₀ for inverse agonism, i.e. inhibition of the basal, constitutive signalling was 5.2±0.7 nM. The stimulatory dose-response curve for ghrelin is indicated as a dotted curve for comparison (see FIG. 3). Panel B: Schild-like analysis, i.e. dose-response curves for ghrelin in the absence and in the presence of [D-Arg¹, D-Phe⁵, D-Trp^{7,9}, Leu¹¹]-Substance P (SP-analog) in three different concentrations; 10⁻⁶ M (diamonds), 10⁻⁷ M (triangles) and 10⁻⁸ M (squares). Note that the basal, constitutive signalling activity of the ghrelin receptor is inhibited by the low doses of the SP-analog without shifting the dose-response-curve for ghrelin to the right, i.e. the compound which in vivo decreases food intake and body weight gain (A. Asakawa et al. 2003) being an inverse agonist without being an antagonist. Experiments were performed in transiently transfected COS-7 cells (20 μg DNA in 75 cm² discs) and mean±S.E. of three to five independent experiments made in duplicate are shown.

FIG. 7 illustrates inverse agonism of a “non-peptide compound”—Zn(II)—through binding to a metal-ion site at the extracellular end of TM-V in the ghrelin receptor. The IC50 for inverse agonism for Zn(II) on basal, constitutive signalling as measured by inositol phosphate turnover (see legend to FIG. 3) in the wild-type ghrelin receptor (open circles) and in the metal ion site engineered receptor (closed circles) was 160±70 μM and 4.3±0.2 μM, respectively. Data are mean±S.E. of three independent experiments made in duplicate.

FIG. 8 illustrates inverse agonism of a small non-peptide “drug-like” compound TM27810 on the ghrelin receptor.

The IC50 for inverse agonism for TM27810 (structure shown in the panel to the right) on the basal, constitutive signalling as measured by inositol phosphate turnover (see legend to FIG. 3) in the ghrelin receptor (closed circles) was 6.5 μM. The inverse agonist inhibition curve for [D-Arg¹, D-Phe⁵, D-Trp^{7,9}, Leu¹¹]-Substance P is shown for comparison.

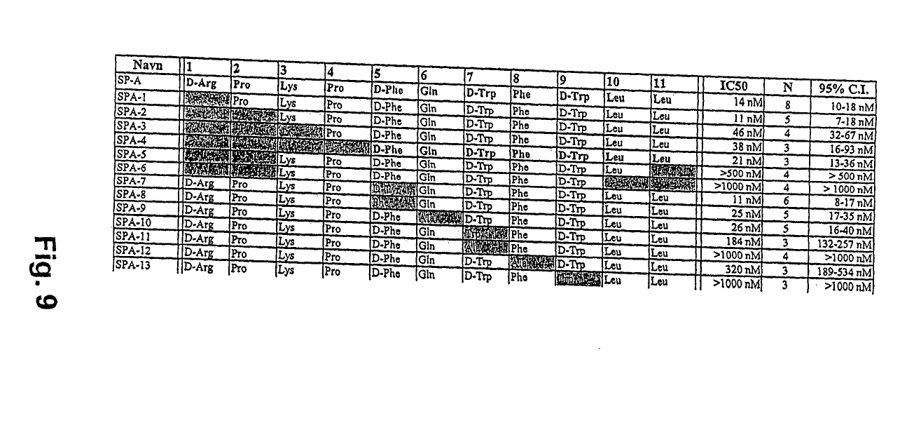

FIG. 9 (Table 1) shows a structure activity relationship (SAR) analysis of the inverse agonist [D-Arg¹, D-Phe⁵, D-Trp^{7,9}, Leu¹¹]-Substance P.

Analogs of [D-Arg¹, D-Phe⁵, D-Trp^{7,9}, Leu¹¹]-Substance P (SP-A) were synthesis and probed for potency as inverse agonists using measurements of inositol phosphate turnover as a read-out (see FIGS. 3 and 6). A series of systematic deletions from the N- and C-terminal ends (SP-A 1 through 6) and a series of single substitutions (or combinations of single substitutions) in the full length SP-A (SP-A 7 through 15) were performed.

The following examples are intended to illustrate the invention without limiting it in any way.

### EXAMPLES

In the following examples are demonstrated that the human ghrelin receptor is characterized by a surprisingly high degree of constitutive signalling activity through multiple signalling pathways and that this activity can be inhibited by peptide as well as non-peptide inverse agonists. In fact, the ligand-independent signalling of the ghrelin receptor is similar to that displayed by one of the most vigorous constitutively active receptors yet reported, the ORF-74 oncogene encoded by human herpes virus 8 (Rosenkilde et al., 1999; Bais et al., 1998). The ligand-independent signalling of the ghrelin receptor has been overlooked until present conceivably due to the fact, that the receptor previously was studied almost exclusively in calcium mobilization assays. In a single preceding publication IP turnover was also employed (Hansen et al., 1999); however, in that study an ultra-short incubation period of only one minute was used—due to the “high noise level” and it was not described as being a reflection of constitutive signalling by the ghrelin receptor. The high constitutive activity of the ghrelin receptor combined with the well established role of the ghrelin hormone/neuropeptides as an important regulator of food intake, energy expenditure and body fat mass opens for novel pharmaco-therapeutic opportunities in developing inverse agonist compounds for the ghrelin receptor for the treatment of, for example obesity. Interestingly, the ghrelin receptor belongs to a small subset of 7TM receptors including the neurotensin receptors and the motilin receptor for which a number of small molecule, non-peptide drug-like ligands previously have been developed—some of which even have been in clinical trials. However, for the ghrelin receptor almost exclusively agonist ligands have as yet been discovered through chemical screening and, importantly inverse agonist ligands have not previously been described.

### Example 1

The Ghrelin Receptor Signals Constitutively Through the Phospholipase C Pathway as Determined in Spontaneous, Ligand-Independent Stimulation of Inositol Phosphate Turnover

In previous studies mobilization of intracellular calcium had almost exclusively been used to monitor the signalling of the ghrelin receptor. However, intracellular calcium is not a good measure for constitutive receptor signalling since—apart from short-lived fluctuations associated with ligand mediated, acute receptor activation—the levels of intracellular calcium is kept constant within a narrow range by a multitude of regulatory mechanisms. Thus, in order to study the ligand independent, spontaneous activity of the ghrelin receptor changes in phospholipase C activity as measured in inositol phosphate turnover was determined in cells transiently transfected with the ghrelin receptor. A convenient way of studying constitutive receptor signalling is to determine the effect of increasing the number of receptors in cells on a relevant intracellular signalling pathway. If the receptor signals spontaneously an increase in ligand-independent signalling will be observed when more and more receptors are expressed in the cells for example by increasing the dose of DNA coding for the receptor in transfected cells. In the present example this is found for the ghrelin receptor in respect of stimulating inositol phosphate turnover.

Material and Methods

Compounds

Ghrelin and [D-Arg¹, D-Phe⁵, D-Trp^{7,9}, Leu¹¹]—Substance P were purchased from Bachem (Bubendorf, Swicherland). A series of analogs of the [D-Arg¹, D-Phe⁵, D-Trp^{7,9}, Leu¹¹]-substance P were prepared through classical Fmoc peptide synthesis by professor Annette Beck-Sickinger. TM27810, 3-[5-(4-Bromo-phenyl)-1-(3-trifluoromethyl-phenyl)-1H-pyrrol-2-yl]-propionic acid (BTPPA) was purchased from Chemical Diversity Labs, Inc.

Molecular Biology

The human ghrelin receptor also called the Growth Hormone Secretagogue receptor (GHS-R) cDNA was cloned by PCR from a human brain cDNA library. The cDNA was cloned into the eukaryotic expression vector pcDNA3 (Invitrogen, Carlsbad, Calif.). Mutations were constructed by PCR using the overlap expression method. The PCR products were digested with appropriate restriction endonucleases, purified and cloned into pcDNA3. All PCR experiments were performed using pfu polymerase (Stratagene, La Jolla, Calif.) according to the instructions of the manufacturer. All mutations were verified by restriction endonuclease mapping and subsequent DNA sequence analysis using an ABI 310 automated sequencer. The cDNA for the negative control, the motilin receptor was provided by Bruce Conklin, The Gladstone Institute, SF and the cDNA for the human herpes virus 8 encoded ORF74 receptor by Mette Rosenkilde from Laboratory for Molecular Pharmacology.

Transfections and Tissue Culture

COS-7 cells were grown in Dulbecco's modified Eagle's medium 1885 supplemented with 10% fetal calf serum, 2 mM glutamine and 0.01 mg/ml gentamicin. Cells were transfected using calcium phosphate precipitation method with chloroquine addition as previously described. HEK-293 cells were grown in D-MEM, Dulbecco's modified Eagle's medium 31966 with high glucose supplemented with 10% fetal calf serum, 2 mM glutamine and 0.01 mg/ml gentamicin. Cells were transfected with Lipofectamine 2000 (Life Technologies).

Phosphatidylinositol Turnover

One day after transfection COS-7 cells were incubated for 24 hours with 5 □Ci of [3H]-myo-inositol (Amersham, PT6-271) in 1 ml medium supplemented with 10% fetal calf serum, 2 mM glutamine and 0.01 mg/ml gentamicin per well. Cells were washed twice in buffer, 20 mM HEPES, pH 7.4, supplemented with 140 mM NaCl, 5 mM KCl, 1 mM MgSO4, 1 mM CaCl2, 10 mM glucose, 0.05% (w/v) bovine serum; and were incubated in 0.5 ml buffer supplemented with 10 mM LiCl at 37□C. for 30 min. The indicated curves were furthermore incubated with adenosine deaminase ADA (200 U/mg, Boeringer Mannheim, Germany) for 30 min in a concentration of 1 U/ml. After stimulation with various concentrations of peptide for 45 min at 37° C., cells were extracted with 10% ice-cold perchloric acid followed by incubation on ice for 30 min. The resulting supernatants were neutralized with KOH in HEPES buffer, and the generated [3H]-inositol phosphate was purified on Bio-Rad AG 1-X8 anion-exchange resin as described. Determinations were made in duplicates.

Calculations

IC50 and EC50 values were determined by nonlinear regression using the Prism 3.0 software (GraphPad Software, San Diego). Values of the dissociation and inhibition constants (Kd and Ki) were estimated from competition binding experiments using the equations Kd=IC50-L and Ki=IC50/(1+L/Kd), where L is the concentration of radioactive ligand.

Results

Determinations of IP accumulation was used as a measure of signalling through the Gq, phospholipase C pathway in COS-7 cells transiently transfected with the human ghrelin receptor. Gene-dosing experiments demonstrated a dose-dependent but ligand-independent increase in IP accumulation in cells expressing the ghrelin receptor as opposed to cells transfected with the empty pcDNA3 vector (FIG. 3 left panel). Since it previously has been shown that adenosine possibly could act as an agonist on the ghrelin receptor and since adenosine perhaps could be produced by the cells used for transfection, we pretreated the cells with adenosine deaminase (ADA). However, ADA did not affect the observed ligand-independent signalling of the ghrelin receptor (FIG. 3, left panel); and—importantly—pretreatment with the same concentration of ADA totally blocked the cAMP accumulation observed upon stimulation of the cells with adenosine conceivably acting through endogenous adenosine receptors expressed on the COS cells (data not shown). An increased production of IP was observed in cells transfected with the ghrelin receptor upon stimulation with 10-6 M ghrelin, which was most clearly observed at the higher levels of receptor expression (FIG. 3, left panel).

That the ghrelin receptor signals with an unusually high degree of constitutive activity, was most clearly demonstrated by comparing its activity to that displayed by its closest homologue, the motilin receptor. In cells transfected with the motilin receptor the ligand independent production of IP was similar to that observed in cells transfected with the empty expression vector, i.e. being 19 and 21%, respectively, of that observed in cells transfected with the ghrelin receptor (FIG. 3, right panel). Upon stimulation with the motilin peptide ligand, IP accumulation reached a level comparable to that observed in cells transfected with the ghrelin receptor after stimulation with the ghrelin agonist (FIG. 3, right panel). In fact, the constitutive, ligand-independent signalling of the ghrelin receptor was comparable to that observed with one of the most well-established highly constitutively active 7TM receptors, the virally encoded ORF74 receptor (FIG. 3, right panel) (14;15).

Thus—this example demonstrates that the ghrelin receptor signals constitutively through the phospholipase C pathway as determined in spontaneous, ligand-independent stimulation of inositol phosphate turnover which is substantiated through the use of the structurally closely related motilin receptor, which in parallel experiments shows no signs of constitutive activity but which signals with a similar strength when exposed to its agonist—the peptide motilin—demonstrating that the expression of the ghrelin and the motilin receptors is similar and that the observed constitutive signalling of the ghrelin receptor is not caused by an increased expression of this receptor.

### Example 2

The Ghrelin Receptor Signals Constitutively Through Multiple Intracellular Pathways as Illustrated by the cAMP Responsive Element (CRE) and the Factor of Activated T Cell (NFAT) Gene Transcription Pathways

The ghrelin receptor is expressed on NPY/AGRP expressing cells in the arcuate nucleus of the hypothalamus, where its stimulatory signalling is supposed to counteract the inhibitory action of for example the Gi coupled Y2 receptors. However, when expressed in heterologous cells it has not been possible to detect any reproducible effect of the ghrelin receptor directly on cAMP production (Gi inhibits cAMP production and it would therefore be expected that the ghrelin receptor should increase cAMP production to have the opposite effect of the Y2 receptor). However, in the present example we demonstrate that the ghrelin receptor signals constitutively through the downstream cAMP responsive element (CRE) pathway (conceivably activated through some intermediate kinase pathway). In fact the high constitutive signalling activity of the ghrelin receptor can be detected in multiple intracellular signalling pathways. In the present example this is further substantiated by measuring the factor of activated T cell (NFAT) gene transcriptional activity in a reporter assay.

Material and Methods (for General Molecular Pharmacological Methods etc. see Example nr. 1)

CRE and NFAT Reporter Assay.

In both reporter assays HEK293 cells (30,000 cells/well) seeded in 96-well plates were transiently transfected. The indicated amounts of receptor DNA were co-transfected with a mixture of pFA2-CREB and pFR-Luc reporter plasmid (PathDetect CREB trans-Reporting System, Stratagene) in case of the CRE reporter assay and in case of the NFAT reporter assay with pNFAT-luc. One day after transfection, cells were treated with the respective ligands in an assay volume of 100 μl medium for 5 hrs. When treated with the ligands cells were maintained in low serum (2.5%) throughout the experiments. The assay was terminated by washing the cells twice with PBS and addition of 100 μl luciferase assay reagent (LucLite, Packard). Luminescence was measured in a TopCounter (Top Count NXT™, Packard) for 5 sec. Luminescence values are given as relative light units (RLU).

Results

The ghrelin receptor signals constitutively through multiple intracellular signalling pathways. Here, this is demonstrated by using two reporter assays for respectively cAMP responsive element (CRE) transcriptional activity and for the factor of activated T cell (NFAT) transcriptional activity. As shown in FIG. 4, panel A, the basal, ligand-independent CRE activity in creased in transiently transfected cells exposed to increasing amounts of DNA coding for the ghrelin receptor. At high doses a subsequent decrease in activity was observed, conceivably due to an over-dosing effect. Addition of a maximal dose of the ghrelin agonist resulted in an even higher CRE activity and demonstrated that the ligand-independent signalling of the ghrelin receptor in this reporter system was between ½ to ¾ of the maximal signalling capacity of the receptor. The homologous motilin receptor (see FIG. 2) displayed no detectable constitutive activity in this assay, but upon stimulation with motilin a strong signal was observed of a magnitude similar to that observed with the ghrelin receptor (FIG. 4, middle panel). As in example nr. 1, the experiments with the motilin (negative-control) receptor demonstrates that the constitutive signalling observed with the ghrelin receptor is not caused by over-expression of this receptor. Like the ghrelin receptor, the virally encoded ORF-74 receptor also signaled with high ligand-independent activity through the CRE pathway with an efficacy, which was even somewhat higher than the maximal efficacy observed for the ghrelin receptor (FIG. 4, right panel). However, as compared to both the motilin receptor (agonist stimulated response) and the ORF-74 receptor (ligand independent response) the gene-dose required for ghrelin receptor to stimulate CREB transcriptional activity was surprisingly almost two orders of magnitude lower. In fact a bell-shaped stimulation was observed with the ghrelin receptor. Thus, the ghrelin receptor in a highly efficient, ligand independent manner stimulates transcriptional activity though the CRE pathway.

As shown in FIG. 5, gene-dosing experiments with the ghrelin receptor also resulted in a ligand independent signalling through the NFAT transcriptional pathway. At high doses the signalling leveled out.

Thus, in the present example gene-dosing experiments demonstrate a dose-dependent but ligand-independent stimulation by the ghrelin receptor through both the CRE and the NFAT pathways indicating that multiple signalling pathways can be used to measure the constitutive activity of the ghrelin receptor and therefore also to monitor the activity f inverse agonists for the ghrelin receptor.

### Example 3

The Constitutive Signalling of the Ghrelin Receptor can be Inhibited Totally by a Potent Inverse Agonist [D-Arg¹, D-Phe⁵, D-Trp^{7,9}, Leu¹¹]-Substance P, Which is Known to be a Low Potency Ghrelin Receptor Antagonist That Can Decrease Food Intake and Body Weight Gain In Vivo

Almost exclusively agonists have been described for the ghrelin receptor. However, a multi-substituted analog of the neuropeptides substance P, [D-Arg¹, D-Phe⁵, D-Trp^{7,9}, Leu¹¹]-Substance P was described as being a low potency ghrelin receptor antagonist (16). In the present example we confirm that this peptide is a low potency antagonist of the ghrelin receptor and describe that it surprisingly is a high potency inverse agonist at this receptor and thereby serve as an example of compounds having a desired profile of being able to selectively eliminate the ligand-independent signalling of the ghrelin receptor, which is believed to be a major driving factor for increased appetite and food intake—nibbling and snacking—in between meals.

Material and Methods

(see Example nr. 1)

Results

The low potency antagonistic effect of [D-Arg¹, D-Phe⁵, D-Trp^{7,9}, Leu¹¹]-Substance P could be confirmed using IP accumulation as a measure of the signalling of the ghrelin receptor, as the substance P analog inhibited the ghrelin stimulated IP accumulation with an EC50 for antagonism of 630 nM. When [D-Arg¹, D-Phe⁵, D-Trp^{7,9}, Leu¹¹]-Substance P was applied to the ghrelin receptor in the absence of ghrelin it was found that the peptide also functioned as a high efficacy, full inverse agonist as it inhibited the spontaneous, ligand-independent signalling in cells transfected with the ghrelin receptor down to the level observed in cells transfected with the empty expression vector (FIG. 5). Surprisingly, the potency of [D-Arg¹, D-Phe⁵, D-Trp^{7,9}, Leu¹¹]-Substance P as an inverse agonist was observed to be 5.2 nM, which is approximately 100-fold higher than the potency of the same peptide when studied as an antagonist against ghrelin (FIG. 5). Thus [D-Arg¹, D-Phe⁵, D-Trp^{7,9}, Leu¹¹]-Substance P is a high potency, high efficacy inverse agonist for the constitutive, ligand-independent signalling of the human ghrelin receptor whereas it functions as a relative low potency antagonist for ghrelin induced signalling.

[D-Arg¹, D-Phe⁵, D-Trp^{7,9}, Leu¹¹]-Substance P is a micromolar antagonist on the NK1 receptor as judged by its ability to block SP induced accumulation of IP in COS-7 cells transiently transfected with the NK1 receptor (data not shown). According to the literature, [D-Arg¹, D-Phe⁵, D-Trp^{7,9}, Leu¹¹]-Substance P is a micromolar antagonist also on for example the bombesin receptor 1. Although the high potency of the [D-Arg¹, D-Phe⁵, D-Trp^{7,9}, Leu¹¹]-Substance P on the ghrelin receptor and its relative specificity, i.e. having nanomolar potency on the ghrelin receptor and micromolar potency on other receptors, strongly indicate that its effect as an inverse agonist on the ghrelin receptor is a specific structurally-based function, a structure-activity analysis of the peptide to substantiate this point and to try to identify a smaller, essential substructure which is responsible for its inverse agonist property, was performed. As shown in Table 1, initially the N-terminal residues were deleted one by one, which demonstrated that residues 1 through 4 could be deleted without any detectable effect on the potency of the peptide as an inverse agonist on the ghrelin receptor. In contrast, deletion of either the last or the two last amino acid residues resulted in a total loss of potency as an inverse agonist (Table 1). This identifies the [DPhe⁵-DTrp^{7,9}] substance P(5-11) as a core structural element which holds all the properties of the original SP analog in respect of being an inverse agonist on the ghrelin receptor. Substitution of the first five residues in the [DPhe⁵-DTrp^{7,9}] substance P(5-11) by either an Ala or a non-D amino acid showed that the two first residue, D-Phe5 and Gln6 were not very important for the function of the peptide as an inverse agonist on the ghrelin receptor as peptides in which DPhe5 was substituted with Gln and Gln6 with Ala had similar potencies as the unmodified peptide. In contrast similar substitutions of DTrp⁷ and DTrp—even just with the corresponding L-amino acids—totally eliminated the activity of the peptide as an inverse agonist. Substitution of Phe8 with Ala resulted in a 30-fold shift of the dose-response curve to the right also showing that the side chain of this residue is important for the overall function of the peptide as an inverse agonist on the ghrelin receptor. It is concluded that the structure activity analysis (SAR) of could be performed on the [D-Arg¹, D-Phe⁵, D-Trp^{7,9}, Leu¹¹]-Substance P peptide in respect of its high potency function as an inverse agonist on the ghrelin receptor and that the core structural unit which is responsible for this function probably is the [DTrp^{7,11}]SP(7-11), although the importance of especially residues 5 is not totally defined by the present library of peptides analogs.

The relatively small functional structural epitope or unit of [D-Arg¹, D-Phe⁵, D-Trp^{7,9}, Leu¹¹]-Substance P indicates that a classical peptide mimetic approach could be applied to design peptoidal and non-peptide ligands for the ghrelin receptor, which would have similar properties as inverse agonists for the ghrelin receptor.

Importantly, [D-Arg¹, D-Phe⁵, D-Trp^{7,9}, Leu¹¹]-Substance P has—after the submission of this patent application—been shown to decrease food intake and body weight gain in both normal and obese mice in vivo (A. Asakawa et al. 2003). When that study was performed it was believed that the substance P analog served as an antagonist for the ghrelin receptor. However, it is demonstrated in the present example that this peptide is a 100-fold selective inverse agonist at the ghrelin receptor. It is therefore concluded that the effects observed in vivo with this peptide shows that an inverse agonist for the ghrelin receptor-will efficiently decrease food intake and body weight even in obese subject. It will be obvious to people knowledgeable in the field that this property will not be limited to the substance P analog or to peptides but will cover inverse agonists for the ghrelin receptor in general.

### Example 4

The Constitutive Signalling of the Ghrelin Receptor can be Inhibited Also by Non-Peptide Inverse Agonists as Illustrated by Zn(II) in a Metal-Ion Site Engineered Ghrelin Receptor and by a Small Non-Peptide Drug-Like Compound in the Wild-Type Ghrelin Receptor.

Example 3 shows that a modified peptide can function as an inverse agonist on the ghrelin receptor. However, due to pharmacokinetic and other reasons peptides are only to a certain extent suitable for use as drugs. In the present example the inverse agonistic effects of non-peptide compounds—Zn(II) and small organic drug-like compounds—on the basal, constitutive activity of the ghrelin receptor is demonstrated.

Material and Methods

(see Example nr. 1)

Results

Metal-ion site engineering has previously been used as a molecular probe for both antagonism, agonism and inverse agonism (Elling et al., 1995; Elling et al., 1999; Rosenkilde et al., 1999). Here we built a metal-ion binding site into the ghrelin receptor by substituting residues V:01 and V:05 with His residues. 125 l-ghrelin bound with normal high affinity to the metal-ion site engineered receptor and ghrelin could stimulate IP turnover with a potency and efficacy as in the wild-type receptor (data not shown). Importantly, as shown in FIG. 7, Zn(II) functioned as a full inverse agonist on the metal-ion site engineered receptor with a potency of 4.3 μM through binding to the two His residues located in an i and i+4 position at the extra-cellular end of TM-V. This demonstrates that the ligand-independent activity of the ghrelin receptor can be blocked through binding of a small ligand to the extracellular end of transmembrane segment V (FIGS. 1 and 7). A similar result has been obtained in the HHV8 encoded constitutively active ORF-74 receptor (Rosenkilde et al., 1999). It should be noted that the experiment with Zn(II) in the metal-ion site engineered ghrelin receptor here only serve to demonstrate that it is possible to totally block the ligand-independent signalling of the receptor through binding of a small molecule ligand—in this case a zinc ion—to the extracellular part of the ghrelin receptor—in this case a slightly modified form with a silent metal-ion site. This is important for the invention since the invention is aimed at small molecule, preferentially non-peptide compounds which will serve as inverse agonists against the receptor and most of these will conceivably as the majority of small molecule drugs in general in 7TM G protein coupled receptor bind in between the extracellular ends of the transmembrane segments. Such compounds do not have to pass the cell membrane but can exert their inverse agonist action at the extracellular part of the receptor. The experiments with Zn(II) demonstrates that it is possible to function as a full inverse agonist through binding to the extracellular part of the ghrelin receptor.

In order to show that drug-like non-peptide compounds also could function as inverse agonists on the ghrelin receptor a small target customized library of selected, commercially available drug-like compounds were- screened for their ability to suppress the constitutive signalling activity of the ghrelin receptor as measured as IP turnover in transiently transfected cells. As an example of positive hits in such a screen compound TM27810 is shown in FIG. 8. TM27810, which is 3-[5-(4-Bromo-phenyl)-1-(3-trifluoromethyl-phenyl)-1H-pyrrol-2-yl]-propionic acid (BTPPA) and can be purchased from Chemical Diversity Labs, is a high efficacy inverse agonist of the ghrelin receptor as it dose-dependently decreases the ligand-independent signalling of this receptor with an IC50 for inverse agonism of 6 iM (FIG. 8). It will be obviously to the person knowledgeable in the field that TM27810 only serve as an example of small non-peptide compounds which are inverse agonists at the ghrelin receptor. It will be obvious to the person knowledgeable in the art that chemical modifications of such a compound or other similar lead compounds can increase their affinity and potency and that compounds with appropriate high potency and appropriate pharmacokinetic properties can be developed on the basis of such lead compounds through well established medicinal chemical approaches.

### REFERENCE LIST

Asakawa A, Inui A, Kaga T, Katsuura G, Fujimiya M, Fujino M A and Kasuga M (2003) Antagonism of Ghrelin Receptor Reduces Food Intake and Body Weight Gain in Mice. Gut 52: pp 947-952.
Asakawa A, Inui A, Kaga T, Yuzuriha H, Nagata T, Ueno N, Makino S, Fujimiya M, Niijima A, Fujino M A and Kasuga M (2001) Ghrelin is an Appetite-Stimulatory Signal From Stomach With Structural Resemblance to Motilin. Gastroenterology 120: pp 337-345.
Bais C, Santomasso B, Coso O, Arvanitakis L, Raaka E G, Gutkind J S, Asch A S, Cesarman E, Gershengorn M C, Mesri E A and Gerhengorn M C (1998) G-Protein-Coupled Receptor of Kaposi's Sarcoma-Associated Herpesvirus Is a Viral Oncogene and Angiogenesis Activator. Nature 391: pp 86-89.
Bednarek M A, Feighner S D, Pong S S, McKee K K, Hreniuk D L, Silva M V, Warren V A, Howard A D, Van Der Ploeg L H and Heck J V (2000) Structure-Function Studies on the New Growth Hormone-Releasing Peptide, Ghrelin: Minimal Sequence of Ghrelin Necessary for Activation of Growth Hormone Secretagogue Receptor 1a. J Med Chem 43: pp 4370-4376.
Bowers C Y, Momany F, Reynolds G A, Chang D, Hong A and Chang K (1980) Structure-Activity Relationships of a Synthetic Pentapeptide That Specifically Releases Growth Hormone in Vitro. Endocrinology 106: pp 663-667.
Bowers C Y, Momany F A, Reynolds G A and Hong A (1984) On the in Vitro and in Vivo Activity of a New Synthetic Hexapeptide That Acts on the Pituitary to Specifically Release Growth Hormone. Endocrinology 114: pp 1537-1545.
Choi K, Roh S G, Hong Y H, Shrestha Y B, Hishikawa D, Chen C, Kojima M, Kangawa K and Sasaki S (2003) The Role of Ghrelin and Growth Hormone Secretagogues Receptor on Rat Adipogenesis. Endocrinology 144: pp 754-759.
Date Y, Murakami N, Toshinai K, Matsukura S, Niijima A, Matsuo H, Kangawa K and Nakazato M (2002) The Role of the Gastric Afferent Vagal Nerve in Ghrelin-Induced Feeding and Growth Hormone Secretion in Rats. Gastroenterology 123: pp 1120-1128.
Elling C E, Nielsen S M and Schwartz T W (1995) Conversion of Antagonist-Binding Site to Metal-Ion Site in the Tachykinin Nk-1 Receptor. Nature 374: pp 74-77.
Elling C E, Thirstrup K, Hoist B and Schwartz T W (1999) Conversion of Agonist Site to Metal-Ion Chelator Site in the Beta(2)-Adrenergic Receptor. Proc Natl Acad Sci USA 96: pp 12322-12327.
Horvath T L, Diano S, Sotonyi P, Heiman M and Tschop M (2001) Minireview: Ghrelin and the Regulation of Energy Balance—a Hypothalamic Perspective. Endocrinology 142: pp 4163-4169.
Howard A D, Feighner S D, Cully D F, Arena J P, Liberator P A, Rosenblum C I, Hamelin M, Hreniuk D L, Palyha O C, Anderson J, Paress P S, Diaz C, Chou M, Liu K K, McKee K K, Pong S S, Chaung L Y, Elbrecht A, Dashkevicz M, Heavens R, Rigby M, Sirinathsinghji D J, Dean D C, Melillo D G, Van Der Ploeg L H and (1996) A Receptor in Pituitary and Hypothalamus That Functions in Growth Hormone Release. Science 273: pp 974-977.
Itoh Z (1997) Motilin and Clinical Application. Peptides 18: pp 593-608.
Kojima M, Hosoda H, Date Y, Nakazato M, Matsuo H and Kangawa K (1999) Ghrelin Is a Growth-Hormone-Releasing Acylated Peptide From Stomach. Nature 402: pp 656-660.
Lawrence C B, Baudoin F M and Luckman S M (2002) Centrally Administered Galanin-Like Peptide Modifies Food Intake in the Rat: a Comparison With Galanin. J Neuroendocrinol 14: pp 853-860.
Nakazato M, Murakami N, Date Y, Kojima M, Matsuo H, Kangawa K and Matsukura S (2001) A Role for Ghrelin in the Central Regulation of Feeding. Nature 409: pp 194-198.
Patchett A A, Nargund R P, Tata J R, Chen M H, Barakat K J, Johnston D B, Cheng K, Chan W W, Butler B, Hickey G and (1995) Design and Biological Activities of L-163,191 (MK-0677): a Potent, Orally Active Growth Hormone Secretagogue. Proc Natl Acad Sci USA 92: pp 7001-7005.
Rosenkilde M M, Kledal T N, Brauner-Osborne H and Schwartz T W (1999) Agonists and Inverse Agonists for the Herpesvirus 8-Encoded Constitutively Active Seven-Transmembrane Oncogene Product, ORF-74. J Biol Chem 274: pp 956-961.
Shimizu-Albergine M, Ippolito D L and Beavo J A (2001) Downregulation of Fasting-Induced CAMP Response Element-Mediated Gene Induction by Leptin in Neuropeptide Y Neurons of the Arcuate Nucleus. J Neurosci 21: pp 1238-1246.
Smith R G, Cheng K, Schoen W R, Pong S S, Hickey G, Jacks T, Butler B, Chan W W, Chaung L Y, Judith F and (1993) A Nonpeptidyl Growth Hormone Secretagogue. Science 260: pp 1640-1643.
Thompson N M, Gill D A, Davies R, Loveridge N, Houston P A, Robinson I C and Wells T (2003) Ghrelin and Des-Octanoyl Ghrelin Promote Adipogenesis Directlyin Vivo by a Mechanism Independent of GHS-R1a. Endocrinology.
Tschop M, Smiley D L and Heiman M L (2000) Ghrelin Induces Adiposity in Rodents. Nature 407: pp 908-913.
Tschop M, Statnick M A, Suter T M and Heiman M L (2002) GH-Releasing Peptide-2 Increases Fat Mass in Mice Lacking NPY: Indication for a Crucial Mediating Role of Hypothalamic Agouti-Related Protein. Endocrinology 143: pp 558-568.
Volante M, Allia E, Fulcheri E, Cassoni P, Ghigo E, Muccioli G and Papotti M (2003) Ghrelin in Fetal Thyroid and Follicular Tumors and Cell Lines: Expression and Effects on Tumor Growth. Am J Pathol 162: pp 645-654.

All documents mentioned herein are incorporated herein by reference in their entirety.

1-29. (canceled)

30. An inverse agonist of a ghrelin receptor.

31. An inverse agonist of claim 30 wherein the inverse agonist is identifiable by a method comprising:
contacting a ghrelin receptor with at least one test compound without the presence of an agonist for the ghrelin receptor, and
measuring any change in the basal activity of the ghrelin receptor
identifying test compounds that decrease the basal activity level of the ghrelin receptor by at least 10%.

32. An inverse agonist of claim 30 wherein the inverse agonistic activity is about 20 μM or less when measured in a phosphatidylinositol turnover assay as described in the Examples.

33. An inverse agonist of claim 30 or 31 wherein the ratio between IC50 for inverse agonism and IC50 for antagonism of the inverse agonist is in a range of from about 1:1000 to about 1:10.

34. An inverse agonist of claim 30 or 31 wherein the inverse agonist is not an antagonist of a ghrelin receptor.

35. An inverse agonist of claim 30 or 31 wherein the inverse agonist is also antagonist of a ghrelin receptor.

36. An inverse agonist of claim 35 wherein the antagonistic activity is 10 μM or less when measured in a phosphatidylinositol turnover assay as described in the Examples.

37. An inverse agonist of claim 35 wherein the ratio between IC50 for inverse agonism and IC50 for antagonism of the inverse agonist is in a range of from about 1:10 to about 1:0.01.

38. An inverse agonoist of claim 30 or 31 wherein the inverse agonist is a peptide.

39. An inverse agonist of claim 30 or 31 wherein the inverse agonist is a non-peptide.

40. An inverse agonist of claim 30 or 31 wherein the inverse agonist is an antibody.

41. A pharmaceutical composition comprising an inverse agonist of claim 30 or 31.

42. A pharmaceutical composition of claim 41 further comprising a pharmaceutical acceptable excipient.

43. A pharmaceutical composition of claim 41 wherein the inverse agonist of the ghrelin receptor is present in an amount sufficient to decrease the basic activity level of the ghrelin receptor with at least 10% as evidenced by an in vitro method described in the Examples.

44. A pharmaceutical composition of claim 41 wherein the composition is adapted for enteral and/or parenteral use.

45. A pharmaceutical composition of claim 41 in the form of a solid, semi-solid or fluid composition.

46. A method for identifying a compound which is an inverse agonist of a ghrelin receptor, the method comprising
contacting a ghrelin receptor with at least one test compound without the presence of an agonist for the ghrelin receptor, and
measuring any change in the basal activity of the ghrelin receptor
identifying test compounds, that decreases the basal activity level of the ghrelin receptor with at least 10%.

47. A method for the preparation of a pharmaceutical composition comprising an inverse agonist of a ghrelin receptor identifiable by a method according to claim 46, the method comprising admixing the inverse agonist with one or more pharmaceutically acceptable excipients.

48. A method for modulating by inverse agonism the activity of a ghrelin receptor in a mammal by contacting the receptor with an inverse agonist of claim 30 or 31.

49. A method for the treatment and/or prophylaxis of diseases caused by feeding disorders, the method comprising administering to a mammal in need thereof an effective amount of an inverse agonist of claim 30 or 31.

50. A method for the treatment and/or prophylaxis of overeating including bulimia, bulimia nervosa, overweight and/or obesity, the method comprising administering to a mammal in need thereof an effective amount of an inverse agonist of claim 30 or 31.

51. A method for treatment of overweight and/or obesity, the method comprising administering to a mammal in need thereof an effective amount of an inverse agonist of claim 30 or 31.

52. A method for the treatment and/or prophylaxis of Syndrome X (metabolic syndrome) or any combination of obesity, insulin resistance, dyslipidemia, impaired glucose tolerance and hypertension, the method comprising administering to a mammal in need thereof an effective amount of an inverse agonist of claim 30 or 31.

53. A method for the treatment and/or prophylaxis of Type II diabetes or Non Insulin Dependent Diabetes Mellitus (NIDDM), the method comprising administering to a mammal in need thereof an effective amount of an inverse agonist according of claim 30 or 31.

54. A method for modifying the feeding behavior of a mammal, the method comprising administering to a mammal in need thereof an effective amount of an inverse agonist of claim 30 or 31.

55. A method for suppression of hunger or reducing energy intake of a mammal, the method comprising administering orally to an animal in need thereof an effective amount of an inverse agonist of claim 30 or 31.

56. method for the reduction of body mass, the method comprising administering to a mammal in need thereof an effective amount of an inverse agonist of claim 30 or 31.

57. A cosmetic method for reducing body weight, the method comprising administering to an animal in need thereof, an effective amount of an inverse agonist of claim 30 or 31.

58. A method of claim 49 further comprising administering an effective amount of an antagonist of a ghrelin receptor.
